(19) United States
(12) Patent Application Publication
(10) Pub. No.: US 2021/0030432 A1
(43) Pub. Date: Feb. 4, 2021

(54) VORTEX CATHETER THROMBOLYTIC SYSTEM AND THROMBOLYTIC METHOD THEREOF

(71) Applicant: National Tsing Hua University, Hsinchu (TW)

(72) Inventors: Chih-Kuang YEH, Hsinchu (TW); Wei-Chen LO, Hsinchu (TW)

(21) Appl. No.: 16/525,648

(22) Filed: Jul. 30, 2019

(51) Int. Cl. A61B 17/22; A61M 25/00

(52) U.S. Cl. CPC A61B 17/2202; A61B 2017/00402; A61M 25/0026

(57) ABSTRACT

A vortex catheter thrombolytic system comprises an ultrasonic transducer; and a probe for transmitting a vortex acoustic field as well as a catheter, wherein the catheter is arranged in the probe for transmitting the vortex acoustic field, is connected to the ultrasonic transducer and is provided with a first inner channel and a second inner channel, the first inner channel is used for delivering drugs, and the second inner channel is used for vortex driving. A thrombolytic method comprises: performing an ultrasonic execution step through the vortex catheter thrombolytic system so as to generate an acoustic vortex; executing a focusing step so as to focus a drug delivery carrier to the center of the acoustic vortex; and executing a manipulation step so as to manipulate the drug delivery carrier to a lesion area.

### BACKGROUND

### Technical Field

The present invention relates to a vortex catheter device, and more specifically relates to a vortex catheter thrombolytic system and a thrombolytic method thereof.

### Related Art

Thrombolytic therapy for general vascular thrombosis mainly relies on drugs, including anticoagulants and thrombolytic agents. A thrombolytic agent, such as a tissue plasminogen activator (Tpa), is taken as an example herein. The Tpa is an anticoagulant that was first approved by the US Food and Drug Administration (FDA) to treat thrombus-induced stroke. However, the Tpa also has serious defects. Thrombolytic drugs have the risk of causing bleeding, the Tpa must be used within hours of the onset of stroke symptoms, the drug effect in the body can not be long-lasting and may cause long-lasting bleeding, and large blood clots can not be dissolved usually. If high-dose thrombolytic drugs are used, it is easy to cause massive bleeding in the body.

Generally, ultrasonic waves refer to generally-focused or non-focused ultrasonic waves. In the technical field of ultrasonic emission, it is a general prior art to generate an ultrasonic focusing effect by phase modulation. In order to reduce the dose of thrombolytic drugs and the risk of bleeding and consider the thrombolytic effect, an interventional catheter is used for directly delivering the thrombolytic agents to the thrombus, and general ultrasonic waves are used for strengthening the drugs to ablate the thrombus so as to strengthen the thrombolytic effect. However, general phase modulation does not have the thrombolytic technology for generating a vortex acoustic field by phase modulation of the present invention.

Based on the above, the present invention adopts phase modulation to generate a vortex acoustic field, and the general phase modulation and the phase modulation of the present invention for generating the vortex acoustic field are two completely different technologies. The probe for transmitting the vortex acoustic field is provided with a piezoelectric patch, the piezoelectric patch comprises a plurality of channels, and the phase difference generated between every two channels is used for generating a vortex of an acoustic channel by the ultrasonic transducer. In brief, although the two technologies are based on phase modulation, the objectives are different.

### SUMMARY

The problem to be solved by the present invention is that the technology for generating a vortex acoustic field through phase modulation makes the present invention superior to the general ultrasonic combined thrombolytic drug technology to improve the efficiency in a thrombolytic method.

The present invention is mainly directed to a vortex catheter thrombolytic system, comprising an ultrasonic transducer; and a probe for transmitting a vortex acoustic field as well as a catheter, wherein the catheter is arranged in the probe for transmitting the vortex acoustic field, is connected to the ultrasonic transducer and is provided with a first inner channel and a second inner channel, the first inner channel is used for delivering drugs, the second inner channel is used for vortex driving, the probe for transmitting the vortex acoustic field is provided with a piezoelectric patch, the piezoelectric patch comprises a plurality of channels, and the phase difference generated between every two channels is used for generating a vortex of an acoustic channel by the ultrasonic transducer.

According to the above objective, the probe for transmitting the vortex acoustic field of the present invention can be connected with the catheter in a magnetic adsorption mode, a buckling mode or a gluing mode.

The probe for transmitting the vortex acoustic field of the present invention comprises at least one piezoelectric patch or comprises multiple array elements (at least four array elements) of piezoelectric patches.

The curvature radius of the piezoelectric patch of the present invention ranges from 1 mm to 300 mm.

The present invention is secondarily directed to a vortex catheter thrombolytic system, comprising an ultrasonic transducer; and a radial probe for transmitting a vortex acoustic field as well as a catheter, wherein the catheter is arranged in the radial probe for transmitting the vortex acoustic field, is connected to the ultrasonic transducer and is provided with a first inner channel and a second inner channel, the first inner channel is used for delivering drugs, the second inner channel is used for vortex driving, the radial probe for transmitting the vortex acoustic field can perform vortex motion at two sides, the radial probe for transmitting the vortex acoustic field is provided with a piezoelectric patch, the piezoelectric patch comprises a plurality of channels, and the phase difference generated between every two channels is used for generating a vortex of an acoustic channel by the ultrasonic transducer.

The present invention is also directed to a thrombolytic method, comprising: performing an ultrasonic execution step through the vortex catheter thrombolytic system so as to generate an acoustic vortex; executing a focusing step so as to focus a drug delivery carrier to the center of the acoustic vortex; and executing a manipulation step so as to manipulate the drug delivery carrier to a lesion area.

According to the above objective, the ultrasonic execution step of the present invention is executed by a pulse generator having a duty cycle of 30% or higher.

The parameters in the ultrasonic execution step of the present invention are as follows: the frequency is 0.5-20 MHz, and the acoustic pressure ranges from 0.1 MPa to 2 MPa.

In order to further understand the objectives, effects, features and structures of the present invention, exemplary embodiments in conjunction with accompanying drawings are illustrated below.

### BRIEF DESCRIPTION OF THE DRAWINGS

FIG. 1 is a schematic diagram of a vortex catheter thrombolytic system with an ultrasonic transducer according to an embodiment of the present invention;

FIG. 2 is a schematic diagram of a vortex catheter thrombolytic system according to an embodiment of the present invention;

FIG. 3 is a schematic diagram of a vortex catheter thrombolytic system according to another embodiment of the present invention;

FIG. 4 is a schematic diagram of a vortex catheter thrombolytic system according to another embodiment of the present invention;

FIG. 5 is a flow diagram of a thrombolytic method according to an embodiment of the present invention;

FIG. 6 is a diagram showing a dissolution rate of a thrombolytic experiment under three experiment conditions according to an embodiment of the present invention.

### DETAILED DESCRIPTION

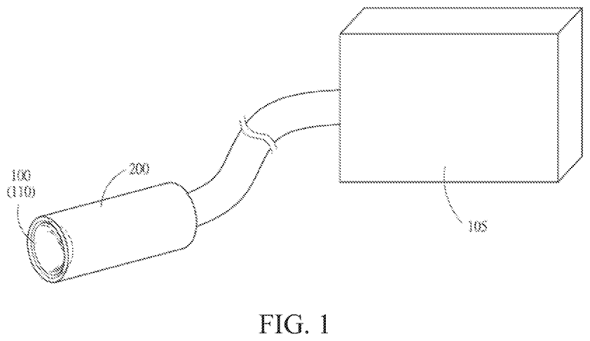

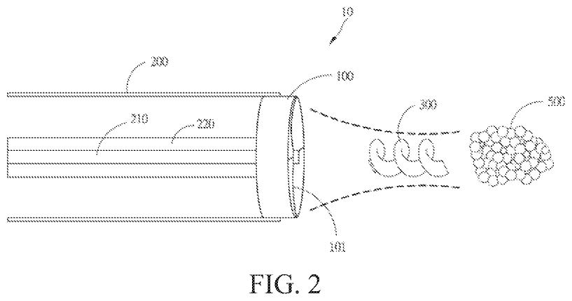

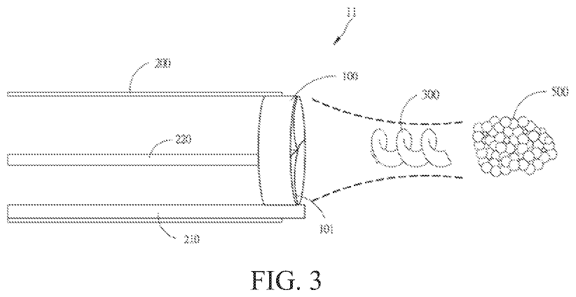

FIG. 1 is a schematic diagram of a vortex catheter thrombolytic system with an ultrasonic transducer according to an embodiment of the present invention, FIG. 2 is a schematic diagram of a vortex catheter thrombolytic system according to an embodiment of the present invention, and FIG. 3 is a schematic diagram of a vortex catheter thrombolytic system according to another embodiment of the present invention. Referring to FIG. 1 and FIG. 2, in an embodiment of the present invention, a vortex catheter thrombolytic system 10 comprises an ultrasonic transducer 105; and a probe 100 for transmitting a vortex acoustic field as well as a catheter 200, wherein the catheter 200 is arranged in the probe 100 for transmitting the vortex acoustic field, is connected to the ultrasonic transducer 105 and is provided with a first inner channel 210 and a second inner channel 220, the first inner channel 210 is used for delivering drugs, the second inner channel 220 is used for vortex driving, the probe 100 for transmitting the vortex acoustic field is provided with a piezoelectric patch 101, the piezoelectric patch 101 comprises a plurality of channels, and the phase difference generated between every two channels is used for generating a vortex 300 of an acoustic channel by the ultrasonic transducer 105.

Referring to FIG. 1 and FIG. 3, in an embodiment of the present invention, a vortex catheter thrombolytic system 11 comprises an ultrasonic transducer 105; and a probe 100 for transmitting a vortex acoustic field as well as a catheter 200, wherein the catheter 200 is arranged in the probe 100 for transmitting the vortex acoustic field, is connected to the ultrasonic transducer 105 and is provided with a first inner channel 210 and a second inner channel 220, the first inner channel 210 is used for delivering drugs, the second inner channel 220 is used for vortex driving, the probe 100 for transmitting the vortex acoustic field is provided with a piezoelectric patch 101, the piezoelectric patch 101 comprises a plurality of channels, and the phase difference generated between every two channels is used for generating a vortex 300 of an acoustic channel by the ultrasonic transducer 105.

Specifically, the ultrasonic transducer 105 can be a pulse generator. More specifically, the ultrasonic transducer 105 can be, but not limited to, a pulse generator based on a field programmable gate array (FPGA). Furthermore, a driving signal transmitted by the ultrasonic transducer 105 can be a square wave signal or a sine wave signal. Although not shown in the figures, an amplifier can be arranged on the ultrasonic transducer 105 to amplify the driving signal.

In an embodiment of the present invention, the piezoelectric patch 101 is made from a lead zirconate titanate material. Furthermore, the piezoelectric patch 101 and a filling and sealing casing are filled with epoxy resin, but the present invention is not limited thereto.

In an embodiment of the present invention, the probe 100 for transmitting the vortex acoustic field can be connected with the catheter 200 in a magnetic adsorption mode, a buckling mode or a gluing mode.

In an embodiment of the present invention, the probe 100 for transmitting the vortex acoustic field comprises at least one piezoelectric patch 101 or comprises multiple array elements (at least four array elements) of piezoelectric patches 101, the piezoelectric patch 101 has a curved shape and is cut into four adjacent channels, and the phase difference generated between every two adjacent channels is used for generating an acoustic vortex.

In an embodiment of the present invention, the curvature radius of the piezoelectric patch ranges from 1 mm to 300 mm.

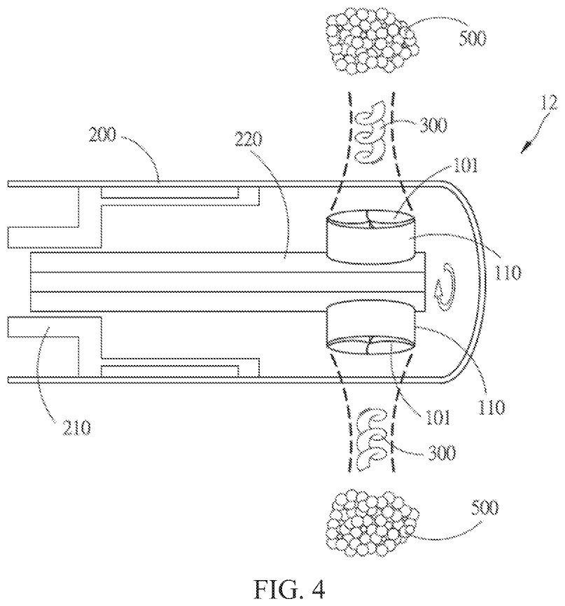

FIG. 4 is a schematic diagram of a vortex catheter thrombolytic system according to another embodiment of the present invention. Referring to FIG. 1 and FIG. 4, in an embodiment of the present invention, a vortex catheter thrombolytic system 12 comprises an ultrasonic transducer 105; and a radial probe 110 for transmitting a vortex acoustic field as well as a catheter 200, wherein the catheter 200 is arranged in the radial probe 110 for transmitting the vortex acoustic field, is connected to the ultrasonic transducer 105 and is provided with a first inner channel 210 and a second inner channel 220, the first inner channel 210 is used for delivering drugs, the second inner channel 220 is used for vortex driving, the radial probe 110 for transmitting the vortex acoustic field can perform vortex motion at two sides so as to increase the vortex driving to the radial direction of blood vessels, the radial probe 110 for transmitting the vortex acoustic field is provided with a piezoelectric patch 101, the piezoelectric patch 101 comprises a plurality of channels, and the phase difference generated between every two channels is used for generating a vortex 300 of an acoustic channel by the ultrasonic transducer 105.

In an embodiment of the present invention, the radial probe 110 for transmitting the vortex acoustic field comprises at least one piezoelectric patch 101 or comprises multiple array elements (at least four array elements) of piezoelectric patches 101, the piezoelectric patch 101 has a curved shape and is cut into four adjacent channels, and the phase difference generated between every two adjacent channels is used for generating an acoustic vortex.

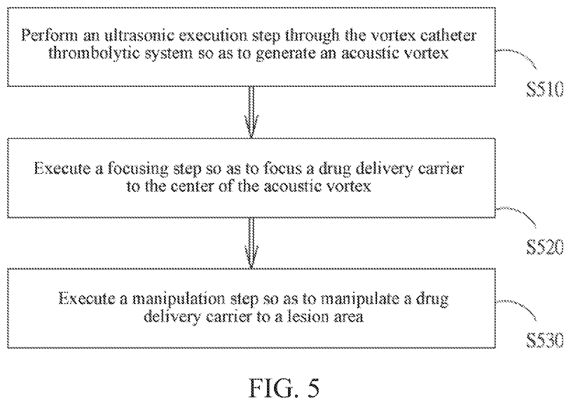

FIG. 5 is a flow diagram of a thrombolytic method according to an embodiment of the present invention. Referring to FIG. 5, in an embodiment of the present invention, a thrombolytic method comprises: performing an ultrasonic execution step through the vortex catheter thrombolytic systems 10, 11 and 12 so as to generate an acoustic vortex; executing a focusing step so as to focus a drug delivery carrier to the center of the acoustic vortex; and executing a manipulation step so as to manipulate the drug delivery carrier to a lesion area.

In an embodiment of the present invention, the ultrasonic execution step is executed by a pulse generator having a duty cycle of 30% or higher.

In an embodiment of the present invention, the parameters in the ultrasonic execution step are as follows: the frequency is 0.5-20 MHz, and the acoustic pressure ranges from 0.1 MPa to 2 MPa.

Referring to FIG. 5, in S510, the ultrasonic execution step is performed through the vortex catheter thrombolytic system so as to generate an acoustic vortex.

Referring to FIG. 5, in S520, the focusing step is executed so as to focus a drug delivery carrier to the center of the acoustic vortex.

Referring to FIG. 5, in S530, the manipulation step is executed so as to manipulate a drug delivery carrier to a lesion area.

Thrombus is a blood clot formed in blood vessels and acts in a blood circulation system to obstruct or block blood flow. When the blood vessels are damaged, in order to avoid blood loss or further damage to the blood vessels caused by blood flow impact, platelets and fibrin in the blood will aggregate to form the blood clot for repairing. However, if the blood clot falls off, it may cause thrombosis to cause embolism.

### An Exemplary Embodiment of the Present Invention

Experiment Method

Preparation of thrombus 500 and simulation of blood flow:

The blood of the human body is divided into plasma and erythrocyte. The plasma accounts for about 55% of the total blood volume, and the erythrocyte accounts for about 45% of the total blood volume. The thrombus can also be prepared by using whole blood. The thrombus in this experiment is prepared from the whole blood of the human body. There may be some slight differences between individuals, and the concentration of calcium chloride can be finely adjusted.

A thrombus preparation formula in the experiment does not require a special ratio and basically comprises 55% of plasma and 45% of erythrocyte. The experiment can also be performed according to the data provided by the medical corporate body Taiwan Blood Services Foundation.

Step 1: plasma, erythrocyte and thrombin 20U were placed in a constant-temperature water tank and heated to 37° C.

Step 2: the plasma, the erythrocyte, the thrombin 20U and calcium chloride (CaCl₂) were sequentially added in proportion.

Step 3: after mixing, a syringe was fixed by a floating pad and placed at the water temperature of 37° C. for 1 h to form a thrombus to be pushed out by the syringe.

Step 4: an Actilyse solution has a function of dissolving the thrombus 500, a solution containing Actilyse (scientific name: Alteplase) was used as a thrombolytic drug (Tpa) in this experiment, the prepared thrombus was added to the thrombolytic drug prepared from the Actilyse solution, then, a solidified state was changed into a flow state, and a peristaltic pump was used for generating a flow environment so as to simulate the blood flow.

TABLE 1
Thrombus preparation and blood flow simulation
experiment material composition

| Experiment material | Parameter |
|---|---|
| Ultrasonic wave | Frequency: 0.5-20 MHz |
| | Acoustic pressure: 1.6 MPa |
| | Duty cycle: 50% |
| Thrombolytic drug | Actilyse |
| Thrombus preparation material | Erythrocyte |
| | Plasma |
| | Thrombin |
| | CaCl₂ |

Thrombolytic experiment method:

At the simulated body temperature of 37° C., three groups of experiment conditions (one control group and two experiment groups) were provided: a thrombolytic drug (Tpa), a general ultrasonic wave combined thrombolytic drug (Tpa), and a vortex acoustic field combined thrombolytic drug (Tpa). In order to compare the difference between the control group and the experiment groups, the experiment conditions were fixed and dissolved for 1 h for comparison, and the thrombolytic effects of all groups were obtained by comparing the residual dose of thrombus dissolution.

Control Group

Thrombolytic drug (Tpa): the thrombolytic drug was added to the thrombus 500, and the residual dose of thrombus dissolution was calculated 1 h later.

Experiment Group 1

General ultrasonic wave combined thrombolytic drug (Tpa): general ultrasonic waves were applied to the thrombus 500 under the conditions that the pressure was 1.6 MPa and the duty cycle was 50%, and the residual dose of thrombus dissolution was calculated 1 h later.

Experiment Group 2

Vortex acoustic field combined thrombolytic drug (Tpa): a vortex acoustic field was applied to the thrombus 500 under the conditions that the pressure was 1.6 MPa and the duty cycle was 50%, and the residual dose of thrombus dissolution was calculated 1 h later.

Thrombolytic Experiment Result

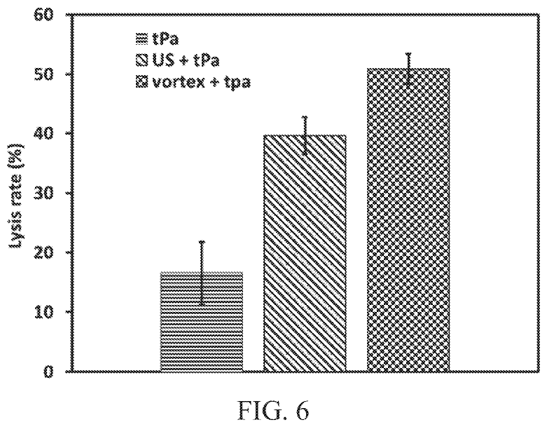

FIG. 6 shows a dissolution rate of a thrombolytic experiment under three experiment conditions.

The dissolution rate of the thrombolytic experiment under three experiment conditions: experiment group 2: the dissolution rate of the vortex acoustic field combined thrombolytic drug (Tpa) was 51%; experiment group 1: the dissolution rate of the general ultrasonic wave combined thrombolytic drug (Tpa) was 40%; and control group: the dissolution rate of the thrombolytic drug (Tpa) was 17%. The results of many experiments show that the thrombus dissolution rate of the experiment group 2 was increased by more than 10% compared with the experiment group 1, and the difference was significant; and the thrombus dissolution rate of the experiment group 2 was increased by more than 34% compared with the control group, and the difference was also significant.

The results of many experiments show that the thrombolytic effect of the vortex acoustic field combined thrombolytic drug (Tpa) was better than that of the general ultrasonic wave combined thrombolytic drug (Tpa), more than 10% of the thrombus dissolution rate was increased, and the difference was significant; and the thrombolytic effect of the vortex acoustic field combined thrombolytic drug (Tpa) was better than that of the single thrombolytic drug (Tpa), more than 34% of the thrombus dissolution rate was increased, and the difference was also significant. The experiments prove that the dissolution rate of the vortex acoustic field combined thrombolytic drug (Tpa) applied to thrombus dissolution was better than that of other prior arts.

The experiments prove that the dissolution rate of the vortex acoustic field combined thrombolytic drug (Tpa) applied to the thrombolytic experiment was better than that of other prior arts.

The probe for transmitting the vortex acoustic field or the radial probe for transmitting the vortex acoustic field of the present invention can enhance the vortex driving effect and achieve the objective of quick thrombus dissolution.

Therefore, the present invention has excellent advancement and practicability in similar products. At the same time, after checking the domestic and foreign technical documents of this kind, it was true that no identical or similar structure or technology exists before the application of the present disclosure. Therefore, the present disclosure should have met the patent requirements of “inventiveness”, “integrated industrial use” and “progressiveness”, and was applied in accordance with the law.

The above embodiments are merely exemplary embodiments of the present invention, and other equivalent structural changes made by the specifications and claims of the present invention are intended to be included in the scope of the claims of the present invention.

What is claimed is:

1. A vortex catheter thrombolytic system, comprising:
an ultrasonic transducer; and
a probe for transmitting a vortex acoustic field as well as a catheter, wherein the catheter is arranged in the probe for transmitting the vortex acoustic field, is connected to the ultrasonic transducer and is provided with a first inner channel and a second inner channel, the first inner channel is used for delivering drugs, the second inner channel is used for vortex driving, the probe for transmitting the vortex acoustic field is provided with a piezoelectric patch, the piezoelectric patch comprises at least one channel, and when there are a plurality of channels, the phase difference generated between every two channels is used for generating a vortex of an acoustic channel by the ultrasonic transducer.

2. The vortex catheter thrombolytic system according to claim 1, wherein the probe for transmitting the vortex acoustic field is connected with the catheter in a magnetic adsorption mode, a buckling mode or a gluing mode.

3. The vortex catheter thrombolytic system according to claim 1, wherein the probe for transmitting the vortex acoustic field comprises at least one piezoelectric patch or comprises multiple array elements (at least four array elements) of piezoelectric patches.

4. The vortex catheter thrombolytic system according to claim 3, wherein the curvature radius of the piezoelectric patch ranges from 1 mm to 300 mm.

5. A vortex catheter thrombolytic system, comprising:
an ultrasonic transducer; and
a radial probe for transmitting a vortex acoustic field as well as a catheter, wherein the catheter is arranged in the radial probe for transmitting the vortex acoustic field, is connected to the ultrasonic transducer and is provided with a first inner channel and a second inner channel, the first inner channel is used for delivering drugs, the second inner channel is used for vortex driving, the radial probe for transmitting the vortex acoustic field can perform vortex motion at two sides, the radial probe for transmitting the vortex acoustic field is provided with a piezoelectric patch, the piezoelectric patch comprises a plurality of channels, and the phase difference generated between every two channels is used for generating a vortex of an acoustic channel by the ultrasonic transducer.

6. A thrombolytic method, comprising:
performing an ultrasonic execution step through the vortex catheter thrombolytic system according to claim 1 so as to generate an acoustic vortex;
executing a focusing step so as to focus a drug delivery carrier to the center of the acoustic vortex; and
executing a manipulation step so as to manipulate the drug delivery carrier to a lesion area.

7. The thrombolytic method according to claim 6, wherein the ultrasonic execution step is executed by a pulse generator having a duty cycle of 30% or higher.

8. The thrombolytic method according to claim 6, wherein the parameters in the ultrasonic execution step are as follows: the frequency is 0.5-20 MHz, and the acoustic pressure ranges from 0.1 MPa to 2 MPa.
